(19) United States
(12) Patent Application Publication
(10) Pub. No.: US 2013/0010258 A1
(43) Pub. Date: Jan. 10, 2013

(54) IMAGING APPARATUS AND IMAGING METHOD

(75) Inventor: Tsutomu Utagawa, Yokohama-shi (JP)

(73) Assignee: CANON KABUSHIKI KAISHA, Tokyo (JP)

(21) Appl. No.: 13/634,649

(22) PCT Filed: Mar. 29, 2011
(86) PCT No.: PCT/JP2011/001876

§ 371 Date: Sep. 13, 2012

(30) Foreign Application Priority Data
Mar. 31, 2010 (JP) 2010-082815

(51) Int. Cl. A61B 3/12

(52) U.S. Cl. 351/205; 351/246

(57) ABSTRACT

An imaging apparatus includes an irradiation unit configured to irradiate an object to be examined with a plurality of measuring beams, a scanning unit configured to perform scanning with the plurality of measuring beams, a specifying unit configured to specify a size of an overlap region where scanning regions of the plurality of measuring beams on the object to be examined overlap one another, and a change unit configured to change one of a center-to-center distance of the scanning regions and a size of each of the scanning regions according to the size of the overlap region.

### TECHNICAL FIELD

The present invention relates to an imaging apparatus and an imaging method and, in particular, to an imaging apparatus and an imaging method for capturing an object to be examined using a plurality of measuring beams.

### BACKGROUND ART

In recent years, an imaging apparatus for capturing a tomographic image of an object to be examined (also referred to as an optical coherence tomographic image) with an optical coherence tomography (OCT) making the advantage of the coherence of low-coherence light (hereinafter referred to as an OCT apparatus) has been used in the medical field, particularly, in the ophthalmologic field. Since the OCT apparatus makes the advantage of the properties of light, the apparatus can acquire a tomographic image with a high resolution on the order of micrometers equivalent to the wavelength of light.

When a subject's eye such as a fundus is measured, the subject's eye often moves, blinks, or involuntarily moves during the measurement. This tends to distort the tomographic image of the subject's eye acquired by the OCT apparatus.

Japanese Patent Application Laid-Open No. 2008-508068 discusses an OCT apparatus which irradiates a pupil (anterior segment) with a plurality of measuring beams to quickly acquire the three-dimensional structure of the pupil. In the OCT apparatus, a scanning region per measuring beam can be reduced to allow the three-dimensional structure to be quickly captured.

### SUMMARY OF INVENTION

It is desirable for an imaging apparatus for capturing a subject's eye using a plurality of measuring beams to improve the controllability of a scanning region of a measuring beam, in particular, the controllability of the size of an overlap region where the scanning regions of a plurality of measuring beams overlap one another, from the viewpoint of user's convenience.

According to an aspect of the present invention, an imaging apparatus includes irradiation means configured to irradiate an object to be examined with a plurality of measuring beams, scanning means configured to perform scanning with the plurality of measuring beams, specifying means configured to specify a size of an overlap region where scanning regions of the plurality of measuring beams on the object to be center-to-center distance of the scanning regions and a size of each of the scanning regions according to the size of the overlap region.

According to another aspect of the present invention, an imaging apparatus includes irradiation means configured to irradiate an object to be examined with a plurality of measuring beams, scanning means configured to perform scanning with the plurality of measuring beams, specifying means configured to specify a center-to-center distance of scanning regions of the plurality of measuring beams on the object to be examined or a size of each of the scanning regions, and change means configured to change one of the center-to-center distance of the scanning regions and the size of each of the scanning regions according to the other of the center-to-center distance of the scanning regions and the size of each of the scanning regions.

According to an exemplary embodiment of the present invention, in an imaging apparatus for capturing an image of a subject's eye using a plurality of measuring beams, setting at least a value of one of the size of each of the scanning regions, the center-to-center distance of the scanning regions, and the size of the overlap region of the scanning regions allows values of the others to be changed. This allows improving the controllability of the size of the scanning region of a measuring beam, in particular, the controllability of the size of the overlap region, thus enhancing user's convenience.

Further features and aspects of the present invention will become apparent from the following detailed description of exemplary embodiments with reference to the attached drawings.

### BRIEF DESCRIPTION OF DRAWINGS

The accompanying drawings, which are incorporated in and constitute a part of the specification, illustrate exemplary embodiments, features, and aspects of the invention and, together with the description, serve to explain the principles of the invention.

FIG. 1 is a block diagram illustrating an OCT apparatus according to a first exemplary embodiment of the present invention.

FIG. 2A is a schematic diagram illustrating a position adjuster at a fiber end according to the first exemplary embodiment.

FIG. 2B is a schematic diagram illustrating the position adjuster at the fiber end according to the first exemplary embodiment.

FIG. 2C is a schematic diagram illustrating the position adjuster at the fiber end according to the first exemplary embodiment.

FIG. 3A is a schematic diagram illustrating a plurality of beams forming an image on a sensor according to the first exemplary embodiment.

FIG. 3B is a schematic diagram illustrating a plurality of beams forming an image on a sensor according to a second exemplary embodiment of the present invention.

FIG. 4A is a schematic diagram illustrating raster scan according to the first exemplary embodiment.

FIG. 4B is a schematic diagram illustrating raster scan according to the first exemplary embodiment.

FIG. 4C is a schematic diagram illustrating raster scan according to the first exemplary embodiment.

FIG. 5 is a flow chart illustrating an imaging method according to the first exemplary embodiment.

FIG. 6A is a graph illustrating the frequency characteristic of a light source according to the first exemplary embodiment.

FIG. 6B is a graph illustrating the output signal of a sensor according to the first exemplary embodiment.

FIG. 7 is a schematic diagram illustrating a position adjuster at a fiber end according to the second exemplary embodiment.

FIG. 8A is a schematic diagram illustrating raster scan according to the second exemplary embodiment.

FIG. 8B is a schematic diagram illustrating raster scan according to the second exemplary embodiment.

FIG. 9 is a schematic diagram illustrating image compositing according to the first exemplary embodiment.

### DESCRIPTION OF EMBODIMENTS

Various exemplary embodiments, features, and aspects of the invention will be described in detail below with reference to the drawings.

An imaging apparatus according to an exemplary embodiment of the present invention can change the center-to-center distance of scanning regions and the size of each scanning region according to the size of an overlap region where scanning regions of a plurality of measuring beams overlap one another. In other words, the center-to-center distance of scanning regions is a distance between positions where an object to be examined such as a subject's eye is irradiated with a plurality of measuring beams. The center-to-center distance of scanning regions can be changed by a moving unit (e.g., a position adjuster 115 at a fiber end) configured to move the fiber end, for example. The size of each scanning region can be changed by a scanning unit (e.g., a scanning mirror 103 for changing a scanning angle) configured to perform scanning with a plurality of measuring beams, for example. Alternatively, an imaging apparatus according to an exemplary embodiment of the present invention can change the size of each scanning region according to the center-to-center distance of scanning regions. Furthermore, an imaging apparatus according to an exemplary embodiment of the present invention can change the center-to-center distance of scanning regions according to the size of each scanning region.

In an imaging apparatus for capturing an image of an object to be examined using a plurality of measuring beams, the controllability of the scanning region of a measuring beam can be improved. More specifically, the change of the size of the overlap region can be controlled with the region where the object to be examined is scanned by all measuring beams (the entire capture region) made substantially constant. This allows changing the size of the overlap region according to the size of a region of interest, such as the macula lutea or the optic papilla of a subject's eye. Scanning with a plurality of measuring beams is performed so that the overlap region becomes equal to the region of interest to average the tomographic images acquired from the overlap region, thereby allowing the acquirement of a high-quality tomographic image in the region of interest. The decrease of the entire capture region may make the position of the region of interest indistinguishable in the entire fundus. The increase of the entire capture region may prolong a capturing time to probably distort the captured image due to the involuntary eye movement of the subject's eye.

An imaging apparatus according to an exemplary embodiment of the present invention can include a specifying unit for specifying at least one of the size of the overlap region, the center-to-center distance of the scanning regions, and the size of the scanning region, from the viewpoint of user's convenience. Furthermore, an imaging apparatus according to an exemplary embodiment of the present invention can include a display control unit for displaying, on a display unit such as a monitor, an image (such as an icon or a slider, for example, anything will do if it can operate a preset function in such a manner that it is clicked or dragged using a cursor displayed on the display unit) having the function of the specifying unit. The display unit may be integrated with the imaging apparatus, be detachable therefrom, or be capable of communicating therewith by wire or wireless.

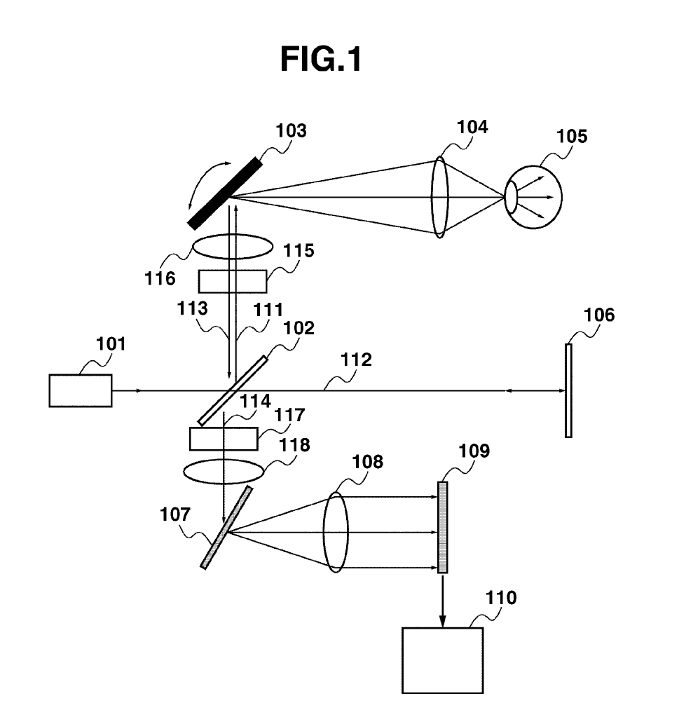

The imaging apparatus according to the present exemplary embodiment is described below with reference to FIG. 1. FIG. 1 is a block diagram illustrating an OCT apparatus according to a first exemplary embodiment of the present invention. The OCT apparatus using four measuring beams as a plurality of measuring beams with which an object to be examined such as a subject's eye is irradiated is described herein. For the sake of simplicity of illustration, four measuring beams are reduced to one beam. Although an optical fiber is used for transferring a plurality of measuring beams in the present exemplary embodiment, the present invention is not limited to this. Although the present exemplary embodiment discusses a spectral domain (SD)-OCT, the present invention is applicable to other types of the OCT (a time-domain (TD)-OCT or a swept-source (SS)-OCT) or a scanning laser ophthalmoscopy (SLO).

Each of the four measuring beams emitted from a light source 101 is split into a reference beam 112 and a measuring beam 111 by a beam splitter 102. A position adjuster 115 (also referred to as a moving unit at a fiber end) at the fiber end (also referred to as an irradiation unit for irradiating an object to be examined with a measuring beam) adjusts the position of the fiber end for emitting the four measuring beams 111. Then, an XY mirror 103 (also referred to as a scanning unit) is irradiated with the measuring beams 111 via a lens 116. The XY mirror 103 reciprocally rotates to raster-scan the fundus of the eye to be observed with the measuring beams 111 according to the command of a CPU for controlling the entire apparatus. An eye 105 to be observed is irradiated with the four measuring beams 111 reflected by the XY mirror 103. The measuring beams 111 with which the eye 105 is irradiated are reflected and scattered at the fundus and returned as return beams 113. After that, the beam splitter 102 is irradiated with the return beams 113 via the lens 116. The return beams 113 are combined with reference beams 112 to become four interfering beams 114 (also referred to as combined beams). The four interfering beams 114 are caused to be incident on a diffraction grating 107 via a lens 118, split by the diffraction grating 107, and imaged by the lens 108 on a line sensor 109. The line sensor 109 is a four-line sensor with four photoelectric conversion element arrays. However, the line sensor 109 may be an area sensor. Four pieces of image information corresponding to the four interfering beams 114 photoelectrically converted by the line sensor 109 are analog-to-digital converted by an image information processing unit 110, and then Fourier-transformed. Furthermore, the four pieces of image information are combined to generate a tomographic image (also referred to as an optical coherence tomographic image) of the fundus of the eye 105. In the present exemplary embodiment, a 10 mm square region of the fundus is vertically and horizontally divided into four regions, and each of the regions is irradiated with one measuring beam 111 to capture an image of the 10 mm square fundus.

The configuration of the light source 101 is described below. The light source 101 is a super luminescent diode (SLD) being a typical low-coherent light source. The wavelength of the light source 101 is 840 nm and the bandwidth thereof is 50 nm. The bandwidth, which affects resolution in the optical axis direction of a tomographic image to be acquired, is an important parameter. The SLD is selected as the type of the light source 101. The light source has only to capable of emitting low-coherent light. An amplified spontaneous emission (ASE) may be used. Near-infrared light is suited as the wavelength of the light source 101 in view of measurement of the eye. The wavelength affects resolution in the horizontal direction of a tomographic image to be acquired, so that the wavelength is desirably as short as possible. Then, a light source with a wavelength of 840 nm is used herein. It is needless to say that another wavelength may be selected depending on a measurement region to be observed.

The reference beam 112 split by the beam splitter 102 is reflected by a mirror 106 and then returned to the beam splitter 102. The optical path length of the reference beam 112 is made equal to that of the measuring beam 111 to allow the reference beam 112 to interfere with the measuring beam 111.

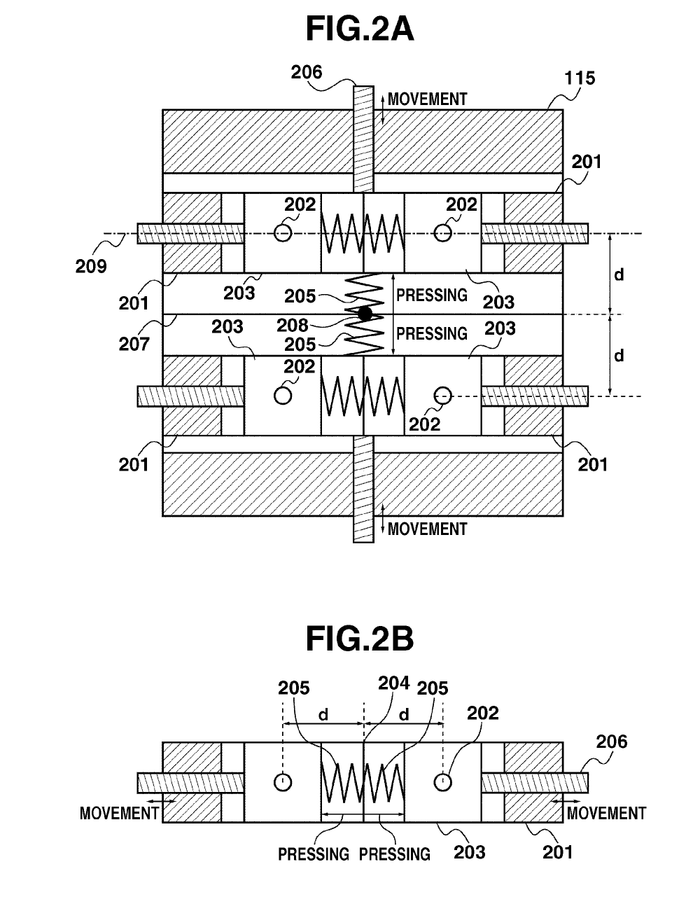

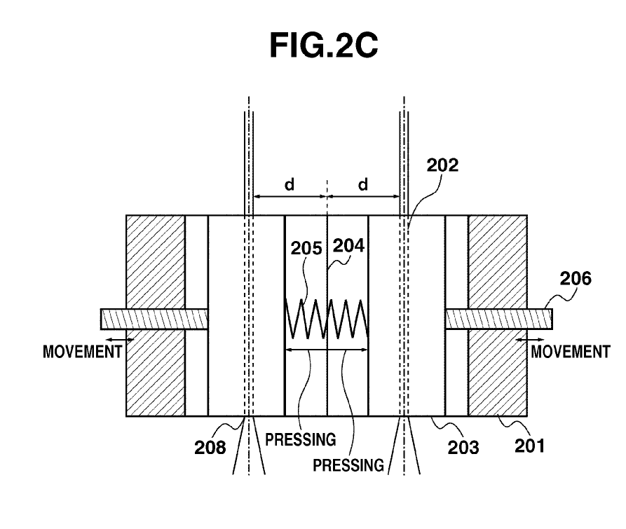

The optical path of the measuring beam 111 is described below. FIGS. 2A to 2C are diagrams illustrating the position adjuster 115 at the fiber end. FIG. 2A is a cross section of the position adjuster 115 at the fiber end as viewed from the side of the optical fiber end. Four optical fibers 202 are fixed with a fiber fixing member 203. The four optical fibers 202 are incorporated into two sets of fiber units 201, two optical fibers for each set. FIG. 2B is a cross section of the fiber unit 201 incorporating the two optical fibers. FIG. 2C is a cross section taken along an alternate long and short dash line 209 in parallel with the optical fibers 202. As illustrated in FIG. 2C, the lens 116 is irradiated from a fiber end 208 with the measuring beam 111 caused to be incident on the position adjuster 115 at the fiber end via the optical fibers 202. The fiber unit 201 is separated by a central wall 204 into two spaces. Each space includes a fiber fixing member 203. The fiber fixing member 203 can be easily moved rightward and leftward in the space of the fiber unit 201 but fixed in the space by the pressure of a screw 206 and a spring 205. The screw 206 is connected with a motor (not illustrated) and can be rotated according to a command from the CPU for controlling the imaging apparatus.

As illustrated in FIG. 2A, the position adjuster 115 at the fiber end is separated by a wall 207 into two spaces. The two fiber units 201 are included in the two spaces of the position adjuster 115 at the fiber end, respectively. The fiber unit 201 can be easily moved upward and downward in the space of the position adjuster 115 at the fiber end but fixed in the space by the pressure of a screw 206 and a spring 205. The screw 206 is connected with a motor (not illustrated) and can be rotated according to a command from the CPU for controlling the imaging apparatus. Normally, the four optical fibers 202 are arranged at equally spaced intervals upward, downward, leftward, and rightward with respect to a center 208 of the position adjuster 115 at the fiber end. A distance d between the four optical fibers 202 can be changed by rotating a motor connected to the screw 206 according to the command of the CPU. In other words, a distance between the four measuring beams 111 with which the fundus is irradiated can be changed by the position adjuster 115 at the fiber end.

The measuring beam 111 split by the beam splitter 102 is incident on the XY mirror 103. For the sake of simplicity of illustration, although the XY mirror 103 is illustrated by one mirror, in practice, an X scan mirror and a Y scan mirror may be adjacently arranged to raster-scan the retina of the eye 105 in the direction perpendicular to the optical axis via a lens 104. The lens 104 converges the measuring beam 111 onto the retina. The measuring beam 111 is caused to be incident on the eye 105 by the above-described optical system to be turned into the return beam 113 by reflection and scattering from the retina of the eye 105.

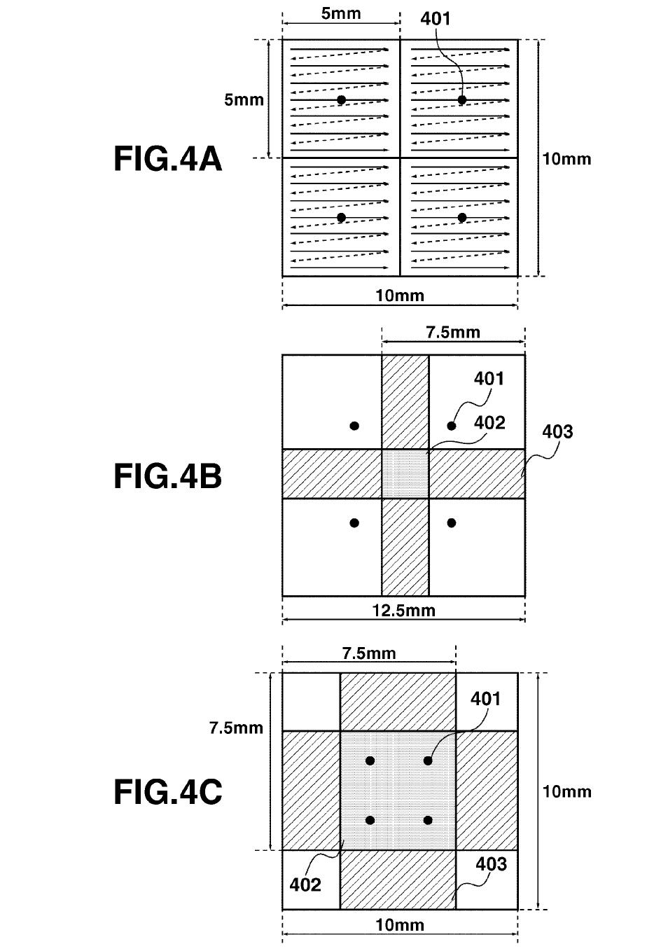

FIGS. 4A to 4C are schematic diagrams illustrating a method for raster-scanning the fundus using the four measuring beams 111. FIG. 4A illustrates how the 10 mm square fundus is vertically and horizontally divided into four portions, which are raster-scanned at a width of 5 mm by the four measuring beams 111. Four dots 401 in FIG. 4A represent centers around which each measuring beam raster-scans each scanning region. In other words, the dot 401 represents a position of irradiation with the measuring beam 111 when the XY mirror 103 lies in the reference position, i.e., in the central position of rotation.

A raster-scan width, i.e., a scanning region on the fundus, can be easily changed by changing an angle at which the XY mirror 103 is rotated via the CPU for controlling the imaging apparatus.

FIG. 4B is a schematic diagram illustrating the scanning region on the fundus in a case where the raster-scan width is increased to 7.5 mm. A center region 402 in FIG. 4B represents a region that is scanned with all of the four measuring beams and is 6.25 mm² in area. A region 403 is scanned with two measuring beams and is 50 mm² in area. Fundus images captured using the four measuring beams are finally combined by the image processing unit 110. The center region 402 has the amount of information being four times as much as image information in a case where raster-scanning is performed without the increase of the scanning region, thus improving an SN ratio. Similarly, the region 403 has the amount of information being twice as much as image information in a case where raster-scanning is performed without the increase of the scanning region.

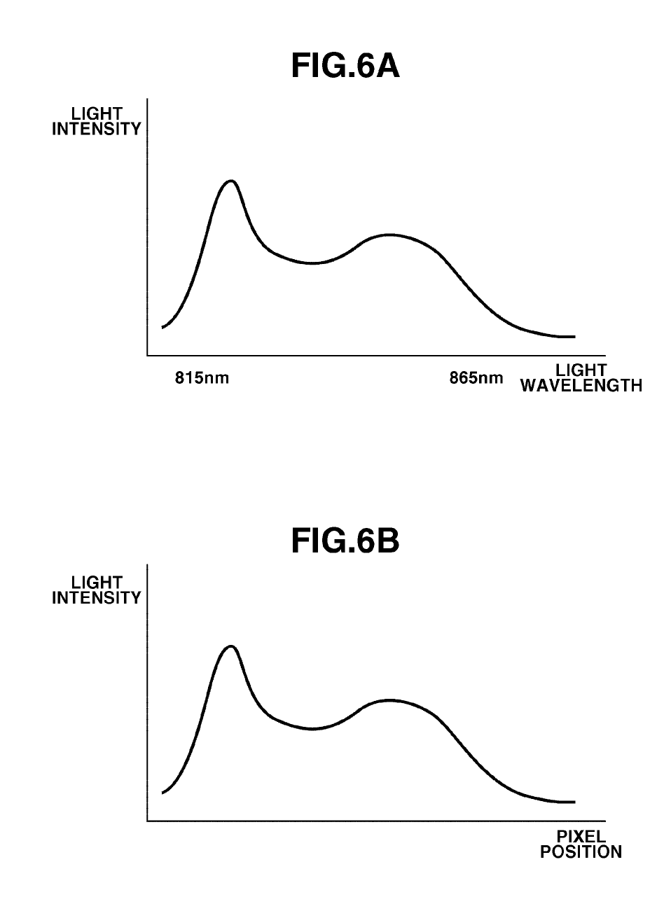

The interfering beam 114 is split by the diffraction grating 107. The interfering beam 114 is split under the wavelength condition similar to the condition for the center wavelength and the bandwidth of the light source. More specifically, the photoelectric conversion element arrays 109-1 to 109-4 (described below) of the line sensor 109 are irradiated with light having a frequency characteristic illustrated in FIG. 6A via the diffraction grating 107 and the lens 108. As illustrated in FIG. 6B, the wavelength of light represented on the abscissa in FIG. 6A becomes a pixel position (0 to 1023) in the photoelectric conversion element arrays 109-1 to 109-4 of the line sensor 109 (on the abscissa in FIG. 6B). A fixing unit 117 at the fiber end fixes the position where the four interfering beams 114 are incident on the diffraction grating 107.

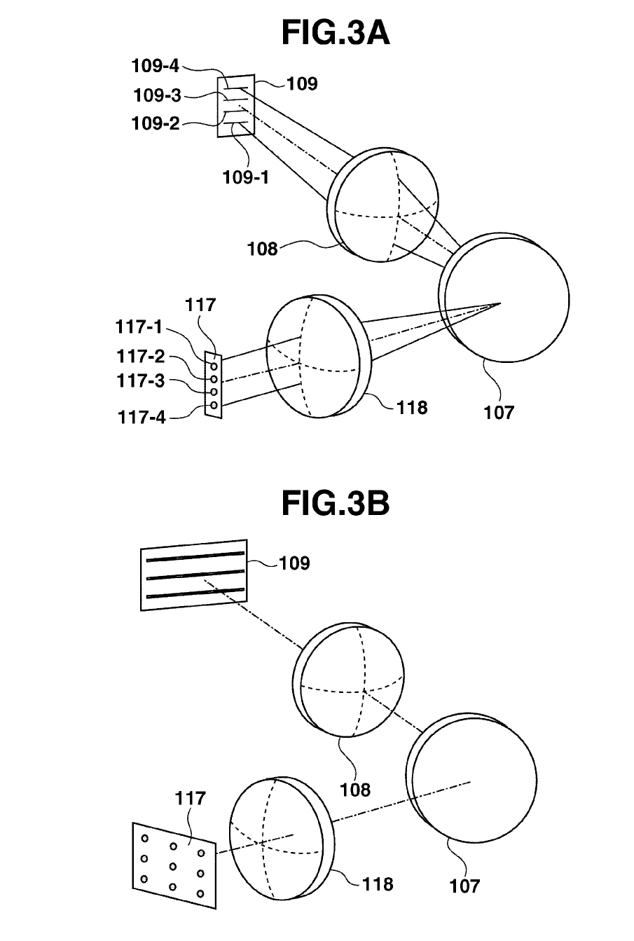

FIG. 3A is a schematic diagram illustrating how the four interfering beams 114 form images on the line sensor 109. Four optical fibers for transferring the four interfering beams 114 respectively are fixed to the fixing unit 117 at the fiber end. The line sensor 109 has four photoelectric conversion element arrays 109-1 to 109-4. The interfering beams 114 emitted from the optical fibers 117-1 to 117-4 fixed to the fixing unit 117 at the fiber end form images on the photoelectric conversion element arrays 109-1 to 109-4, respectively, via the lens 118, the diffraction grating 107, and the lens 108.

The operation of each of the above-described units is controlled by a CPU not illustrated in FIG. 1.

As described above, the width of raster scan of the measuring beam 111 is increased to allow improving the image quality in the center region. Furthermore, a distance between positions where the fundus is irradiated with the measuring beams 111 is changed by the position adjuster 115 at the fiber end along with the increase of width of the raster scan described above, thereby allowing the realization of improvement in image quality by a slight increase in capturing time.

FIG. 4C illustrates the scanning region on the fundus in a case where the width of the raster scan is increased from 5 mm to 7.5 mm and the positions of the four fiber ends are moved by 2.5 mm vertically and horizontally in the center direction by the position adjuster 115 at the fiber end. As is the case with FIG. 4B, the region 402 represents a region that is scanned with all of the four measuring beams and is 25 mm² in area. A region 403 is scanned with two measuring beams and is 50 mm² in area.

The width of the raster scan is increased by 1.5 times from 5 mm to 7.5 mm to increase the scan time, i.e., the capturing time, to 2.25 times being the square of 1.5. However, the region having the amount of information being four times as much as image information accounts for ¼ of the total region, which is very effective for high image-quality capturing.

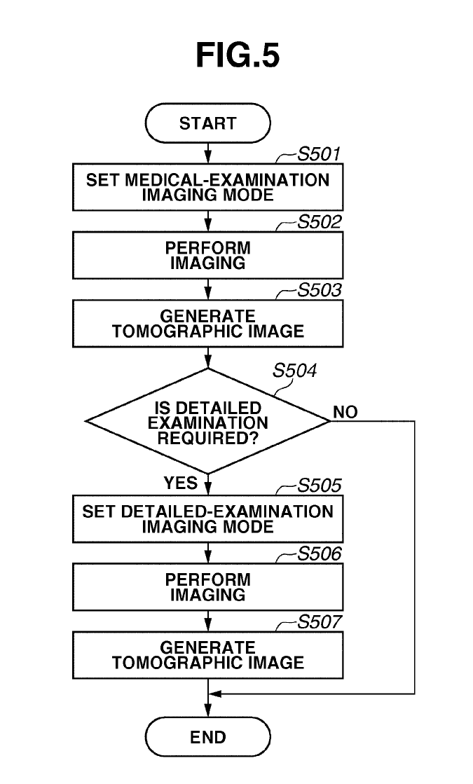

The operation according to the present exemplary embodiment in the OCT apparatus with the above configuration is described below with reference to the flow chart in FIG. 5. The following is a description in a case where the fundus of the object to be measured is captured for medical examination and, if it is determined that disease is suspected from an image for medical examination, a high image quality capturing is conducted.

In step S501, a first imaging mode for medical examination is set. More specifically, as illustrated in FIG. 4A, the 10 mm square region of the fundus is both vertically and horizontally divided into four portions and the position adjuster 115 at the fiber end and the XY mirror 103 are set and controlled so that the measuring beam 111 raster-scans each of the 5 mm square regions without overlapping. In step S502, in the first imaging mode set in step S501, the fundus image of the object to be measured is raster-scanned with the measuring beam 111 to capture the fundus image. In step S503, the image information processing unit 110 subjects the four outputs of the line sensor 109 to Fourier transformation and image compositing to generate a tomographic image of the fundus.

In step S504, the CPU displays the tomographic image of the fundus generated in step S503 on a display device such as a display panel to determine whether the disease of the eye of the object to be measured is suspected. A medical doctor may visually examine the tomographic image of the fundus displayed on the display device to determine whether disease is suspected. Alternatively, a computer may analyze the tomographic image of the fundus to determine whether disease is suspected. If it is determined that the disease of the eye of the object to be measured is not suspected (NO in step S504), measurement is ended. If it is determined that the disease of the eye of the object to be measured is suspected (YES in step S504), then in step S505, a second imaging mode for detailed examination is set. More specifically, a motor (not illustrated) connected to the position adjuster 115 at the fiber end is controlled using the CPU to change the position of irradiation with the measuring beam 111 when the XY mirror 103 lies in the reference position, i.e., in the central position of rotation. As illustrated in FIG. 4B, the four measuring beams 111 are moved upward, downward, leftward, and rightward by 0.625 mm to the center direction.

The angle of rotation of the XY mirror 103 is then changed to change the width of raster scan. More specifically, setting for controlling the XY mirror 103 is provided so that the width at which each measuring beam 111 raster-scans the fundus becomes equal to 6.25 mm. The above description is based on the assumption that the disease of the eye of a patient to be suspected lies in the center of the fundus image. If the disease of a patient to be suspected does not lie in the center of the fundus, the position of a fixation lamp used to change the orientation of an eye of the object to be measured may be changed. In step S506, the fundus image of the object to be measured is raster-scanned with the measuring beam 111 to capture the fundus image. In step S507, the image information processing unit 110 subjects the four outputs of the line sensor 109 to Fourier transformation and image compositing to generate a tomographic image of the fundus, and then, the capturing of fundus image of the object to be measured is ended.

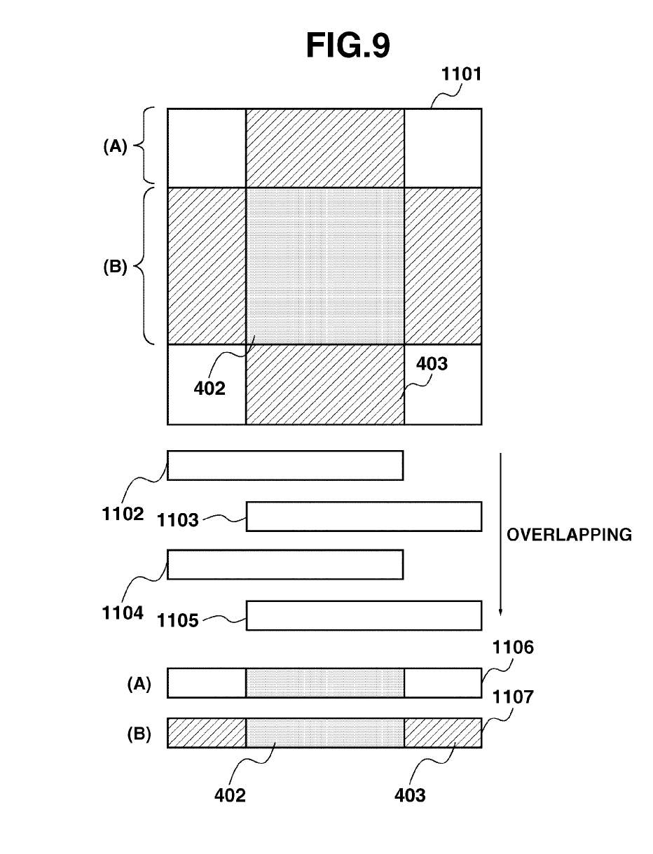

FIG. 9 is schematic diagram illustrating image compositing in the present exemplary embodiment. In FIG. 9, reference numerals 1102 to 1105 represent a state where the scanning region 1101 scanned with the measuring beams 111, as illustrated in FIG. 4C, is viewed upward. The term “image compositing” refers to the arithmetic average of the regions captured with the measuring beams 111 for each pixel. Reference numeral 1106 denotes a state where the region represented by part (A) of the scanning region 1101 is arithmetically averaged. Reference numeral 1107 denotes a state where the region represented by part (B) of the scanning region 1101 is arithmetically averaged. More specifically, as illustrated in FIG. 4C, the amount of information about the image in the center region 402 captured in step S506 is four times as much as that of information captured in the medical examination mode in step S502, the amount of information in the region 403 is twice as much as that of information, and a tomographic image of the fundus generated in step S507 is high in image quality.

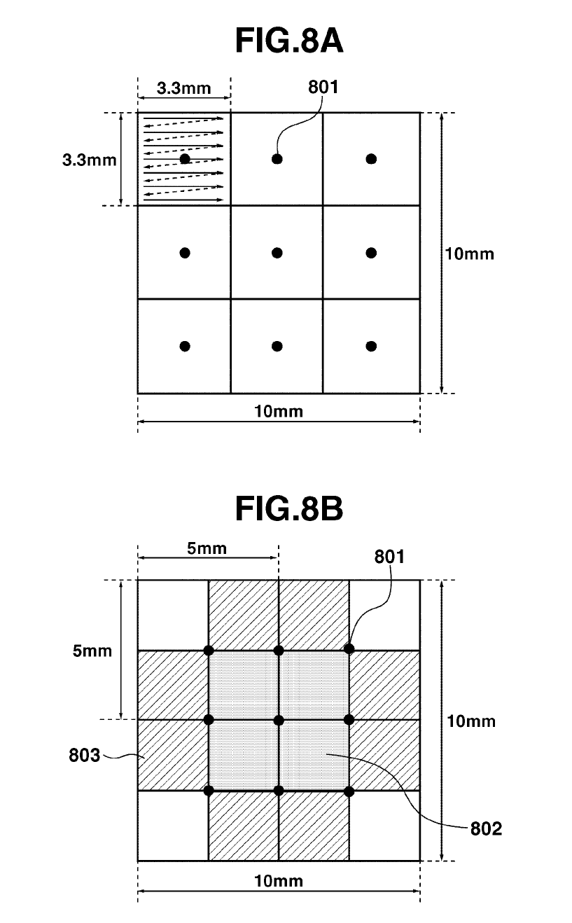

In the first exemplary embodiment, the method for capturing a tomographic image of the fundus using 2*2=4 measuring beams has been described. In a second exemplary embodiment, the method for capturing a tomographic image of the fundus using 3*3=9 measuring beams is described below. FIGS. 8A and 8B are schematic diagrams illustrating the raster scan of the fundus using nine measuring beams. FIG. 8A illustrates how the 10 mm square fundus is vertically and horizontally divided into nine regions and the fundus is raster-scanned at a width of 3.3 mm with the nine measuring beams 111. Nine dots 801 in FIG. 8A represent centers around which each measuring beam raster-scans each scanning region. In other words, the dot 801 represents a position of irradiation with the measuring beam 111 when the XY mirror 103 lies in the reference position, i.e., in the central position of rotation.

FIG. 8B is a schematic diagram illustrating the scanning region on the fundus in a case where the raster-scan width is increased to 5 mm. A center region 802 in FIG. 8B represents a region that is scanned with four measuring beams out of nine and is 25 mm² in area. A region 803 is scanned by two measuring beams and is 50 mm² in area. Fundus images captured using the nine measuring beams are finally combined by the image processing unit 110. The center region 802 has the amount of information being four times as much as image information in a case where raster-scanning is performed without the increase of the scanning region, thus improving an SN ratio. Similarly, the region 803 has the amount of information being twice as much as image information in a case where raster-scanning is performed without the increase of the scanning region.

The width of the raster scan is increased by 1.5 times from 3.3 mm to 5 mm to increase the scan time, i.e., the capturing time, to 2.25 times being the square of 1.5. However, the region having the amount of information being four times as much as image information accounts for ¼ of the total region, which is very effective for high image-quality capturing.

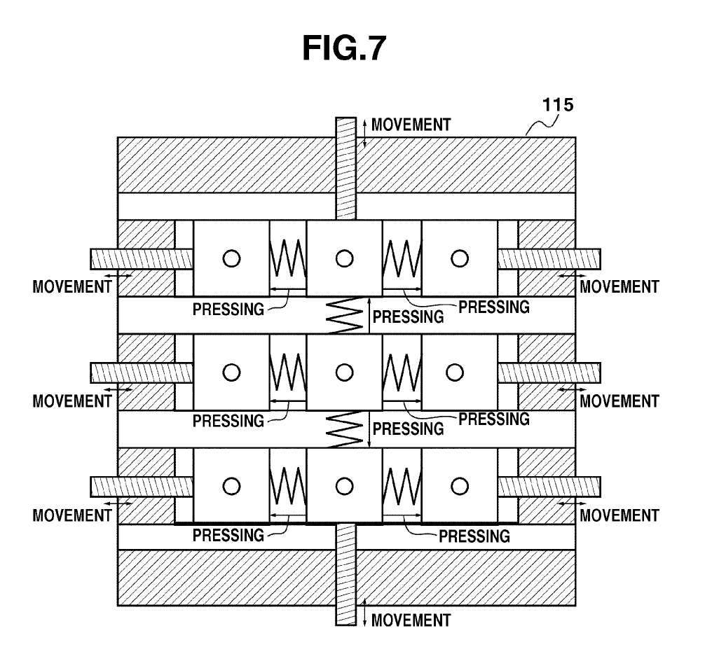

In the above exemplary embodiment, as illustrated in FIG. 7, the position adjuster 115 at the fiber end is configured to be able to adjust nine fiber ends. Furthermore, as illustrated in FIG. 3B, the line sensor is configured to irradiate three photoelectric conversion element arrays of a three-line sensor with three split interfering beams side by side. The block diagram and the operation sequence chart do not need to be changed with the change of the number of measuring beams, so that description is omitted.

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2010-082815 filed Mar. 31, 2010, which is hereby incorporated by reference herein in its entirety.

1. An imaging apparatus comprising:
scanning means configured to perform scanning with a plurality of measuring beams on an object;
specifying means configured to specify a size of an overlap region where scanning regions of the plurality of measuring beams on the object overlap one another; and
change means configured to change at least one of a center-to-center distance of the scanning regions and a size of each of the scanning regions according to the size of the overlap region.

2. (canceled)

3. The imaging apparatus according to claim 1, wherein the change of the center-to-center distance of the scanning regions is a change of a distance between positions where the object is irradiated with the plurality of measuring beams.

4. The imaging apparatus according to claim 3, further comprising irradiation means configured to irradiate the object with the plurality of measuring beams,
wherein the irradiation means includes a plurality of fiber ends configured to irradiate the object with the plurality of measuring beams, and
wherein the change means includes moving means configured to move the plurality of fiber ends.

5. The imaging apparatus according to claim 1, wherein the scanning means includes a common scan mirror of the plurality of measuring beams, and
wherein the change of the size of each of the scanning regions is a change of scanning angle of the scanning means.

6. The imaging apparatus according to claim 1, further comprising display control means configured to display an image having a function of the specifying means on display means.

7. The imaging apparatus according to claim 1, further comprising acquisition means configured to acquire an optical coherence tomographic image of the object based on a plurality of combined beams in which a plurality of return beams from the object irradiated with the plurality of measuring beams is combined with a plurality of reference beams corresponding to the plurality of measuring beams respectively.

8. An imaging method comprising:
performing scanning with a plurality of measuring beams on an object;
specifying a size of an overlap region where scanning regions of the plurality of measuring beams on the object overlap one another; and
changing at least one of a center-to-center distance of the scanning regions and a size of each of the scanning regions according to the size of the overlap region.

9. (canceled)

10. The imaging method according to claim 8, further comprising acquiring an optical coherence tomographic image of the object based on a plurality of combined beams in which a plurality of return beams from the object irradiated with the plurality of measuring beams is combined with a plurality of reference beams corresponding to the plurality of measuring beams respectively.

11. A computer-readable medium storing a program for causing a computer to execute the imaging method according to claim 8.

12. The imaging apparatus according to claim 7, wherein the object is an eye, and
wherein the acquisition means averages at least two optical coherence tomographic images in the overlap region and acquires the optical coherence tomographic image in at least one region of a macula, an optic papilla and a disease of a fundus of the eye.

13. The imaging apparatus according to claim 1, wherein the object is an eye, and
wherein the change means changes at least one of a center-to-center distance of the scanning regions and a size of each of the scanning regions according to a size of at least one region of a macula, an optic papilla and a disease of a fundus of the eye.

14. The imaging apparatus according to claim 1, wherein the change means changes at least one of a center-to-center distance of the scanning regions and a size of each of the scanning regions after the scanning means performs the scanning with the plurality of measuring beams on the object such that the scanning regions do not overlap each other.
